(19) United States
(12) Patent Application Publication
(10) Pub. No.: US 2026/0335073 A1
(43) Pub. Date: Oct. 29, 2026

(54) TISSUE-BASED PROSTHETIC HEART VALVES WITH ANTI-CALCIFICATION PROPERTIES AND METHODS

(71) Applicant: Boston Scientific Scimed, Inc., Maple Grove, MN (US)

(72) Inventors: Laura McKinley, Minneapolis, MN (US); Emily Ann Growney, Galway City, Galway (IE); Rachel Burke, Oranmore, Galway (IE); Christopher Evan Korenczuk, Maple Grove, MN (US); Jami L. McLaren, Plymouth, MN (US)

(21) Appl. No.: 19/658,110

(22) Filed: Apr. 24, 2026

Related U.S. Application Data
(60) Provisional application No. 63/796,142, filed on Apr. 28, 2025.

(51) Int. Cl. A61F 2/24; A61F 2/00; A61L 2/082; A61L 2/206; A61L 27/36; A61L 103/05; A61L 103/15

(52) U.S. Cl. CPC A61F 2/2418; A61F 2/0095; A61F 2/2415; A61F 2/2427; A61L 2/082; A61L 2/206; A61L 27/3604; A61L 27/3641; A61L 2103/05; A61L 2103/15; A61L 2400/02

(57) ABSTRACT

Embodiments herein relate to prosthetic heart valves with anti-calcification properties. In an embodiment, a prosthetic valve system with anti-calcification properties is included having a prosthetic valve including a frame and a plurality of valve leaflets coupled to the frame. The valve leaflets can include an animal tissue with from 15% to 50% by weight water, from 20% to 70% by weight glycerol, and less than 0.01 percent by weight or substantially no glutaraldehyde. The system can include a package with the valve disposed within an interior volume of the package without any glutaraldehyde therein. The animal tissue can be less than or equal to 0.012 inches thick in a sterilized state as disposed within the package and can exhibit anti-calcification properties that are greater than otherwise similar tissue that has not been sterilized within the package. Other embodiments are also included herein.

### CROSS-REFERENCE TO RELATED APPLICATION

This application claims the benefit of U.S. Provisional Application No. 63/796,142 filed Apr. 28, 2025, the content of which is herein incorporated by reference in its entirety.

### FIELD

Embodiments herein relate to prosthetic heart valves with anti-calcification properties.

### BACKGROUND

Heart function can be significantly impaired when a heart valve is not functioning properly. For example, when the heart valve is unable to close properly, the blood within a heart chamber can regurgitate, or leak backwards through the valve. When the heart valve is unable to open properly, forward blood flow (e.g. systolic blood flow) can be impaired.

Valve malfunction can be treated by replacing a diseased valve with a bioprosthetic valve. Minimally invasive methods of valve replacement, such as transcatheter aortic valve replacement (TAVR), generally involve the use of delivery catheters that are delivered through arterial passageways or other anatomical routes into the heart to replace the diseased valve with an implantable prosthetic heart valve. Leaflets of such valves have been formed from various materials including synthetic materials and animal tissues.

Calcification of replacement heart valves represents a significant concern in the long-term performance of bioprosthetic valve implants. Calcification refers to the deposition of calcium, typically in the form of calcium salts, within the tissue matrix of the valve, primarily affecting the flexible valve leaflets. This biological phenomenon can lead to the stiffening and thickening of the leaflet tissues, thereby impairing their mechanical function. As a result, calcification can compromise the hemodynamic functionality of the heart valve, leading to impaired leaflet mobility and eventually to stenosis or regurgitation. Such degenerative changes can necessitate additional medical interventions, including potential valve replacement. Consequently, reducing or inhibiting calcification is a key target in the development of materials and treatment strategies for bioprosthetic heart valves.

### SUMMARY

Embodiments herein relate to prosthetic heart valves with anti-calcification properties. In a first aspect, a prosthetic valve system with anti-calcification properties can be included having a prosthetic valve. The prosthetic valve can include a frame, and a plurality of valve leaflets, wherein the plurality of valve leaflets can be coupled to the frame. The valve leaflets can include an animal tissue. The animal tissue can include from 15% to 50% by weight water, from 20% to 70% by weight glycerol, and less than 0.01 percent by weight glutaraldehyde. The system can further include a package, wherein the prosthetic valve can be disposed within an interior volume of the package without glutaraldehyde. The animal tissue can have an average thickness less than or equal to 0.012 inches thick in a sterilized state as disposed within the package. The animal tissue can exhibit anti-calcification properties that can be greater than otherwise similar tissue that has not been sterilized within the package.

In a second aspect, in addition to one or more of the preceding or following aspects, or in the alternative to some aspects, can further include a delivery catheter.

In a third aspect, in addition to one or more of the preceding or following aspects, or in the alternative to some aspects, the animal tissue can be porcine pericardium.

In a fourth aspect, in addition to one or more of the preceding or following aspects, or in the alternative to some aspects, the prosthetic valve can further include an aortic valve.

In a fifth aspect, in addition to one or more of the preceding or following aspects, or in the alternative to some aspects, the prosthetic valve can further include a transcatheter aortic valve.

In a sixth aspect, in addition to one or more of the preceding or following aspects, or in the alternative to some aspects, the prosthetic valve can further include an inner skirt, wherein the inner skirt can be attached to the frame, and wherein the inner skirt can be formed of an animal tissue, the animal tissue can include from 15% to 50% by weight water, from 20% to 70% by weight glycerol, and less than 0.01 percent by weight glutaraldehyde.

In a seventh aspect, in addition to one or more of the preceding or following aspects, or in the alternative to some aspects, the prosthetic valve can further include an outer skirt, wherein the outer skirt can be attached to the frame, and wherein the outer skirt can be formed of an animal tissue, the animal tissue can include from 15% to 50% by weight water, from 20% to 70% by weight glycerol, and less than 0.01 percent by weight glutaraldehyde.

In an eighth aspect, in addition to one or more of the preceding or following aspects, or in the alternative to some aspects, wherein free aldehydes of the animal tissue can be uncapped.

In a ninth aspect, in addition to one or more of the preceding or following aspects, or in the alternative to some aspects, the animal tissue can be less than or equal to 0.008 inches thick in a sterilized state as disposed within the package.

In a tenth aspect, in addition to one or more of the preceding or following aspects, or in the alternative to some aspects, the animal tissue exhibits deposition of less than 35,000 micrograms of calcium per gram of tissue after 12 weeks in a calcifying solution.

In an eleventh aspect, a method of making a heart valve with anti-calcification properties can be included. The method can include fixing tissue with a glutaraldehyde solution, attaching the tissue to a frame to form valve leaflets, dehydrating the tissue, packaging the heart valve, and sterilizing the heart valve.

In a twelfth aspect, in addition to one or more of the preceding or following aspects, or in the alternative to some aspects, the method can further include rinsing the tissue to remove glutaraldehyde prior to dehydrating the tissue.

In a thirteenth aspect, in addition to one or more of the preceding or following aspects, or in the alternative to some aspects, dehydrating the tissue includes soaking the tissue in a glycerol and saline solution, and drying the tissue in an oven.

In a fourteenth aspect, in addition to one or more of the preceding or following aspects, or in the alternative to some aspects, sterilizing the heart valve includes at least one selected from the group consisting of ethylene oxide (EO) sterilization and irradiation, wherein irradiation takes place in an inert gas atmosphere to reduce oxidative effects from radiation.

In a fifteenth aspect, in addition to one or more of the preceding or following aspects, or in the alternative to some aspects, sterilization includes ethylene oxide (EO) sterilization with a maximum temperature of 50 degrees Celsius or X-ray irradiation with dosage of 15 to 60 kGy.

In a sixteenth aspect, in addition to one or more of the preceding or following aspects, or in the alternative to some aspects, sterilizing the heart valve reduces a thickness of the leaflet tissue by at least 5 percent.

In a seventeenth aspect, in addition to one or more of the preceding or following aspects, or in the alternative to some aspects, the tissue exhibits anti-calcification properties that can be greater than otherwise similar tissue that can have not been sterilized after dehydration and packaging.

In an eighteenth aspect, in addition to one or more of the preceding or following aspects, or in the alternative to some aspects, the tissue can be porcine pericardium.

In a nineteenth aspect, in addition to one or more of the preceding or following aspects, or in the alternative to some aspects, the heart valve can be an aortic valve.

In a twentieth aspect, in addition to one or more of the preceding or following aspects, or in the alternative to some aspects, the tissue exhibits deposition of less than 35,000 micrograms of calcium per gram of tissue after 12 weeks in a calcifying calcium phosphate solution.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures(FIGS.), in which:

FIG. 1 is a schematic view of a prosthetic heart valve within a human body in accordance with various embodiments herein.

FIG. 2 is a view of a valve frame in accordance with various embodiments herein.

FIG. 3 is an end view of a prosthetic heart valve in accordance with various embodiments herein.

FIG. 4 is an end view of a prosthetic heart valve in accordance with various embodiments herein with the leaflets in a closed position.

FIG. 5 is an end view of a prosthetic heart valve in accordance with various embodiments herein with the leaflets in an open position.

FIG. 6 is a schematic view of a delivery catheter in accordance with various embodiments herein.

FIG. 7 is a schematic view of a prosthetic heart valve in a package in accordance with various embodiments herein.

FIG. 8 is a flow chart depicting a method in accordance with various embodiments herein.

FIG. 9 is a chart in accordance with the example herein illustrating reduction in calcification of tissues treated in accordance with embodiments herein.

FIG. 10 is a chart illustrating measured ultimate tensile stress (UTS) values of tissues treated in accordance with embodiments herein.

FIG. 11 is a chart illustrating measured strain values at 1MPa of tissues treated in accordance with embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, calcification of replacement heart valves represents a significant concern in the long-term performance of bioprosthetic valve implants. However, it has been discovered that dehydrated, sterilized, and thinned valve tissue prepared in accordance with embodiments herein surprisingly exhibits improved calcification resistance and biocompatibility as well as providing other advantages.

In specific, the combination of the dehydration process herein along with the sterilization process herein offers a significant reduction in calcification. The combination of these processes results in a structural environment less conducive to the deposition of calcium salts.

Further, the outcome of dehydration in combination with the sterilization process herein is the creation of a thinner valve tissue, which contributes to the ease of loading the valve onto delivery catheters. The reduced thickness enables more efficient crimping onto the catheter, which is an advantage in reducing procedural complexities and can contribute to a smoother catheter insertion and deployment journey through the vasculature.

Furthermore, the dehydration and sterilization processes herein confer enhanced tensile strength to the tissue. Tensile strength is a critical factor for the durability and longevity of heart valve prostheses, given the continuous mechanical stresses experienced within the cardiac environment. By fortifying the structural integrity of the thinned tissue, the valve is better equipped to withstand the repetitive opening and closing cycles without incurring material fatigue or failure.

By way of example, in an embodiment herein, a prosthetic heart valve system is included that is designed to prevent calcification. The heart valve system includes a heart valve that is built with a supporting frame and multiple leaflets attached to it. These valve leaflets use animal tissue that comprises between 15% and 50% water by weight, 20% to 70% glycerol by weight, and contains less than 0.01% glutaraldehyde by weight after treatment herein. The valve can be placed inside a package with no glutaraldehyde present in the package environment. Sterilization processes as executed in the context of methods herein has been found to reduce the thickness of the tissue. In some embodiments, the average tissue thickness after a sterilization process herein is less than 0.012, 0.010, 0.009, 0.008, 0.0075, 0.007, 0.0065, 0.006, or even 0.0057 inches (or less than 0.305, 0.254, 0.229, 0.203, 0.178, 0.165, 0.152, or 0.145 millimeters), or an amount falling within a range between any of the foregoing. It will be appreciated, however, that starting tissue thickness may vary depending on the animal as well as the type of tissue. As such, in various embodiments, the decrease in thickness associated with sterilization herein may be at least about 1, 3, 5, 7, 10, 15, 20, 25, 30, 35, 40, 45, 50 percent or more, or an amount falling within a range between any of the foregoing. The processed tissue exhibits superior anti-calcification effects compared to similar tissue that did not undergo the combination of dehydration and sterilization herein.

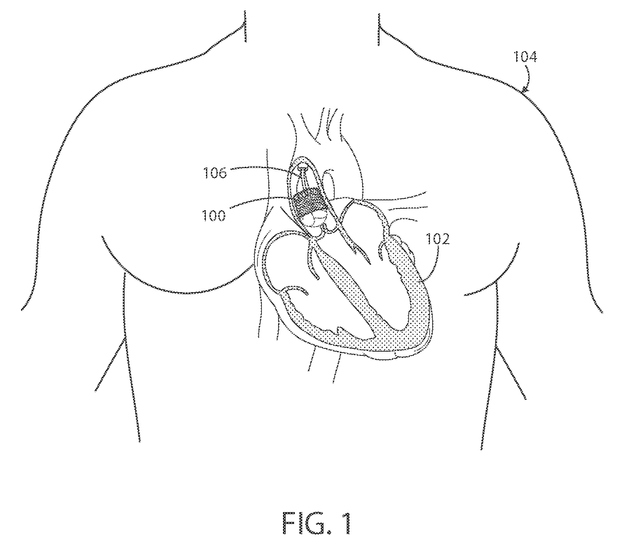

Referring now to FIG. 1, a schematic view of a prosthetic heart valve 100 within a heart 102 of a human body 104 is shown in accordance with various embodiments herein. The heart has four heart valves: a pulmonary valve, a tricuspid valve, an aortic valve and a mitral valve. The heart valves allow blood to pass through the heart and into major blood vessels connected to the heart, for example, the aorta and pulmonary artery. The prosthetic heart valve 100 of FIG. 1 can be surgically implanted or delivered through blood vessels using a delivery device or catheter 106. The delivery catheter 106 can be inserted into a femoral, subclavian, or an aortic incision during a transcatheter aortic valve replacement (TAVR) procedure. In various embodiments, the delivery catheter 106 can include a transfemoral delivery catheter 106. Once inserted, the delivery catheter 106 can deliver the prosthetic heart valve 100 to the desired location within the anatomy and release the heart valve 100 at a desired implantation site. Although FIG. 1 shows prosthetic heart valve 100 replacing an aortic valve, in some cases, prosthetic heart valve 100 can be a replacement for another type of heart valve (e.g., a pulmonary valve, a mitral valve or a tricuspid valve). In some examples the heart valve is specifically a TAVI (transcatheter aortic valve implantation) valve.

A heart valve replacement system can include a heart valve 100 and a delivery catheter 106. In various embodiments, the delivery catheter 106 and the heart valve 100 are disposed within a sterile package after manufacturing and before end use. Aspects of sterilization are described in greater detail below. In various embodiments, the heart valve 100 can be disposed around a portion of the delivery catheter 106. In various embodiments, the heart valve 100 or a portion thereof can be fully crimped onto the delivery catheter 106.

Heart valves herein can include a frame. The frame of a prosthetic heart valve can function as a structural support element that maintains the geometric integrity and dimensional stability of the valve assembly. The frame can provide a scaffold to which the valve leaflets are attached, ensuring correct orientation and functional dynamics during cardiac cycles. This structural support facilitates the opening and closing of the valve leaflets efficiently.

The frame also facilitates secure anchoring of the valve within the native valve annulus or the anatomical position of the intended deployment, resisting displacement due to hemodynamic forces. Its design can ensure optimal contact with the implantation site to minimize paravalvular leakage, which is important for maintaining hemodynamic efficiency. Depending on the material and construction, such as the use of shape-memory alloys, the frame can be engineered to expand upon deployment from the delivery catheter, conforming to the surrounding tissue for a precise fit.

Additionally, the frame can be configured to withstand cyclical loading without significant fatigue over prolonged periods of time, thus ensuring long-term durability of the heart valve. This is particularly critical given the continuous mechanical stresses exerted by the cardiac environment. The integration of features such as stabilization arches and valve supports, such as struts, within the frame can further enhance stability, providing a reliable base that preserves the valve's functional form over repeated cardiac cycles.

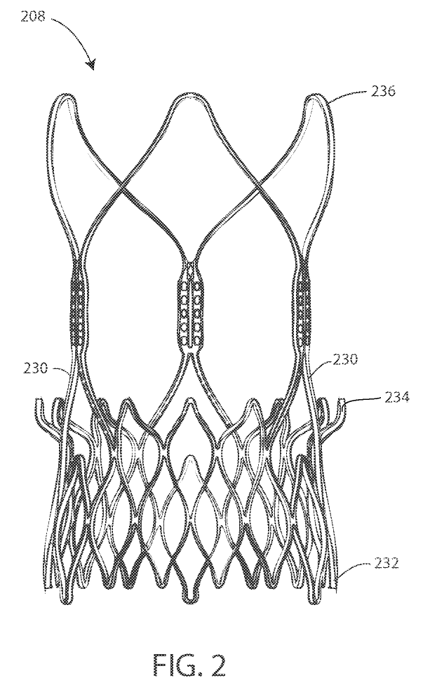

Referring now to FIG. 2, a view of an exemplary frame is shown in accordance with various embodiments herein. The frame 208 can include a lower crown 232. The frame 208 can include an upper crown 234. The frame 208 can include a plurality of stabilization arches 236. In various embodiments, the frame 208 can include a self-expanding stent.

The frame 208 can include one or more valve supports, such as struts 230. The struts 230 can be rods, bars, plates, or other projections that define a framework or lattice structure. In some embodiments, the frame can define open spaces between the frame struts 230. However, in other embodiments the frame may not include such open spaces.

The frame 208 can be formed of many different materials. In some embodiments, the frame 208 can include a polymer, a metal, a ceramic, a composite, or the like. In various embodiments, the metal can include or be an elemental metal or an alloy. In various embodiments, the elemental metal can include or be aluminum or titanium. In various embodiments, the alloy can include or be stainless steel, nitinol or a ferrous metal. In some embodiments, the frame 208 can include shape-memory material, such as a shape-memory alloy.

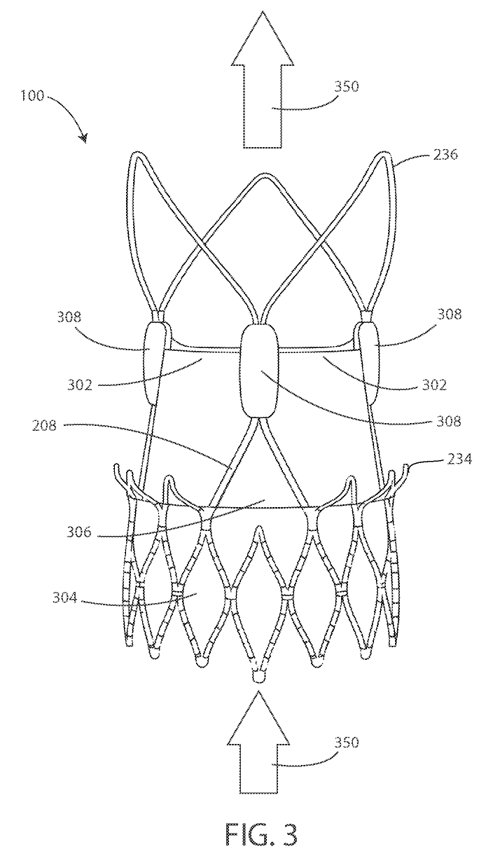

In some embodiments, an inner skirt (such as shown in FIG. 3) can be coupled to the lower crown 232 and/or the upper crown 234 and/or the valve supports. In other embodiments, the inner skirt is only coupled to the upper crown 234. In some embodiments, the outer skirt can be coupled to the lower crown 232 and/or the upper crown 234. In other embodiments, the outer skirt is only coupled to the lower crown 232. In some embodiments, valve leaflets can be coupled to the frame, such as to the valve supports, at a position that is at or just below the stabilization arches 236 and above the upper crown 234. In some embodiments, the valve leaflets can be coupled to the inner skirt, such as in the region of the struts 230 and the upper crown 234.

In the context of heart valve prosthetics, the utilization of natural tissue, such as porcine or bovine tissue, presents significant advantages in replicating the biomechanical functions of native heart valves. These natural tissues possess intrinsic flexibility and elasticity, which are important for the dynamic opening and closing actions required in heart valve mechanics. Porcine pericardial tissue, in particular, is favored due to its anatomical and physiological similarity to human tissue, enabling it to perform similarly under the repetitive stresses and pressures encountered in the cardiac environment. Leveraging the inherent properties of porcine pericardial tissue, bioprosthetic heart valves can mimic the hemodynamic performance of natural valves more closely than purely synthetic alternatives.

The integration of natural tissue within the construction of heart valve leaflets can also offer potential benefits in terms of blood compatibility and reduction of thrombogenic risks. The natural endothelial-like surface of animal tissues can potentially promote favorable interaction with blood components, contributing to the minimization of clot formation.

To attach natural tissue, such as porcine tissue, to a heart valve frame to form various components of the heart valve, the tissue is typically first prepared and sized for the intended application. Specifically, the porcine tissue, which may include pericardial tissue, can be sectioned into appropriately sized pieces for creating leaflets, inner skirts, outer skirts, and/or other structural elements. To form valve leaflets, the natural tissue can be precisely cut into geometric configurations that allow for effective coaptation and dynamic movement within the heart valve frame. These configurations are optimized to achieve a balance between flexibility and structural integrity.

The process of attaching the porcine tissue to the heart valve frame can involve a secure fixation method such as suturing, adhesive bonding, or another technique. When configuring the leaflets, the root edge of each leaflet can be aligned with the designated areas on the frame, typically along valve supports or struts that provide the necessary support and tension during mechanical cycles.

The porcine tissue can also be adapted to form inner and outer skirts of the heart valve. The inner skirt can be configured to adhere closely to the internal contour of the valve frame, thus requiring the porcine tissue to be tailored and fixed at points that correspond to structural elements such as the upper and lower crowns or valve supports. This specific attachment ensures the inner skirt maintains a barrier against backflow when the valve is in a closed configuration. For the outer skirt, the porcine tissue can be applied to the abluminal surface of the frame, providing an additional sealing interface between the valve and the surrounding heart tissue, thereby reducing the potential for paravalvular leakage.

Referring now to FIG. 3, a view of a prosthetic heart valve 100 is shown in accordance with various embodiments herein. The heart valve 100 includes a frame 208 and one or more valve leaflets 302. Blood moves through the valve 100 in the direction of arrows 350. In various embodiments, the heart valve 100 can also include a skirt, such as an outer skirt 304 and/or an inner skirt 306. In various embodiments, the leaflets and/or the skirt can include an animal tissue, for example, pericardial tissue, as described further below.

In various embodiments, the inner skirt 306, if provided, can be disposed on a luminal surface of the frame 208. The inner skirt 306 can direct blood flowing through the valve 100. The inner skirt 306 can ensure the blood flows through the central lumen of the valve 100 and does not flow around the leaflets 302 in a closed configuration during diastolic pressure, such as when the valve is configured as an aortic valve. A portion of each leaflet, such as along the root edge, may be coupled to or sutured to the inner skirt 306.

In various embodiments, the valve 100 can include an outer skirt 304 disposed on an abluminal surface of the frame 208. The abluminal surface of the frame 208 can be a surface of the frame 208 that is external to a central lumen thereof. An outer skirt can be disposed between the frame 208 and the vessel wall when the valve is implanted to aid in preventing blood from flowing around the valve 100 (e.g. to prevent so-called para-valve leakage).

In various embodiments that include both the inner skirt 306 and outer skirt 304, the skirts 306, 304 can be coupled together, such as sutured together, along one or more continuous lines of attachment (not shown), optionally through apertures of the frame.

In various embodiments, commissural mounting tabs of the leaflets 302 can be sutured to and/or passed through apertures in the frame 208. In some embodiments, commissures or attachment members 308 can be disposed over the areas where the leaflets 302 are attached to the frame 208.

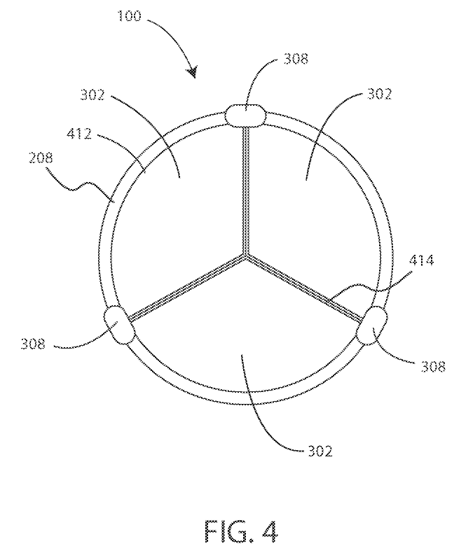

Referring now to FIG. 4, an end view of the heart valve 100 is shown with the leaflets 302 in a closed position. In various embodiments, the heart valve 100 includes a frame 208 (shown in a simplified form in this view for ease of illustration). The frame 208 can define a central lumen which, in some embodiments, can be substantially cylindrical. The side of the frame 208 and other components facing the central lumen can be referred to as the luminal surface or luminal side. The opposite side of the frame 208 and other components (e.g., facing away from the central lumen) can be referred to as the abluminal surface or abluminal side. In various embodiments, the frame 208 can have a substantially circular cross-section. In other embodiments, the frame 208 can have a non-circular, such as a D-shaped, cross-section. In some embodiments, a non-circular frame can be used to repair a mitral valve or another non-circular valve in the body.

The heart valve 100 can also include a plurality of valve leaflets 302, such as two or three leaflets 302. The heart valve 100 can include a coaptation region, such as where one or more leaflets 302 meet to close the valve 100 or separate to open the valve 100. In various embodiments, the valve leaflets 302 are coupled directly or indirectly to the frame 208, e.g. for support by the frame 208. For example, the valve leaflets 302 can be coupled at least in part with attachment members 308. The valve leaflets 302 can include a root edge 412, such as an edge of the leaflet 302 that is coupled to or adjacent to the frame 208. The valve leaflets 302 can also include a coaptation edge 414, such as an edge that aligns with an edge of an adjacent valve leaflet 302. The coaptation edge 414 can be movable relative to the root edge 412 to coapt with the coaptation edges 414 of the other leaflets 302. The coaptation edges 414 of the leaflets 302 move into coaptation with one another in a closed position to substantially restrict fluid from flowing past the valve 100. Specifically, the leaflets 302 can coapt to fill up or close the central lumen of the valve 100.

In various embodiments, the inner diameter of the central lumen can be at least 10 millimeters (mm) and not more than 50 mm. In various embodiments, the inner diameter of the central lumen can be at least 15 mm and not more than 40 mm. In various embodiments, the inner diameter of the central lumen can be at least 20 mm and not more than 35 mm.

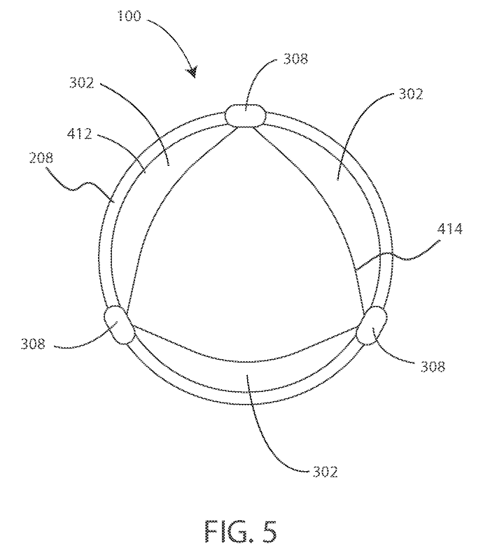

Referring now to FIG. 5, an end view of the heart valve 100 is shown with the leaflets 302 in an open position. As with FIG. 4, FIG. 5 shows heart valve 100, frame 208, leaflets 302, attachment members 308, root edge 412, and coaptation edges 414. However, in this case the leaflets 302 have moved into an open position allowing a flow of blood to pass there through.

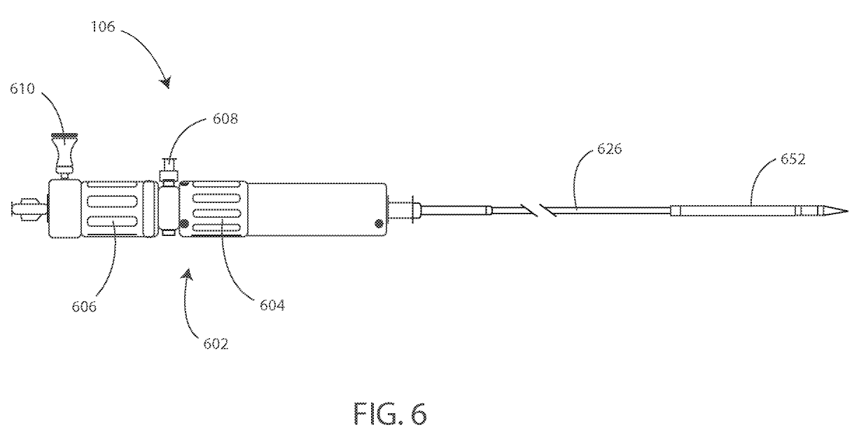

The deployment of prosthetic heart valves utilizing delivery catheters necessitates precision in delivery, positioning, and subsequent deployment within the cardiac anatomy. A delivery catheter, as illustrated in FIG. 6, serves as a vehicle for maneuvering the heart valve into the desired anatomical location. The catheter is introduced through a percutaneous entry point, such as the femoral artery, and advanced through the vascular network under imaging guidance to ensure accurate navigation towards the diseased native valve site.

Upon approach, the catheter, equipped with a constraining sheath and, in some cases, a balloon or other expansion mechanism, maintains the heart valve in a crimped state for streamlined advancement. Once the target site is reached, fine adjustments to catheter position can be made by the operator under fluoroscopic or echocardiographic control, ensuring that the prosthetic valve is aligned accurately with the annulus of the native valve.

In some embodiments, deployment proceeds by retracting the constraining sheath in a controlled manner, optionally using balloon inflation if required, to expand the heart valve frame to its functional diameter, securely seating the valve within the anatomical annulus. In instances where the prosthetic valve employs a self-expanding frame, the removal of the constraining sheath initiates an automatic expansion, facilitated by any embedded shape-memory materials. The valve is thus permitted to engage with the surrounding cardiac structures, and anchoring can be facilitated by the frame's contour conformity and applied radial forces to ensure positional stability and minimize the risk of paravalvular leakage. The precise alignment enabled by this delivery process supports optimal leaflet coaptation for effective restoration of hemodynamic flow dynamics.

Referring now to FIG. 6, a schematic view of a delivery catheter 106 is shown in accordance with various embodiments herein. As discussed above, in some embodiments, the heart valve replacement system can include a delivery catheter 106. The delivery catheter 106 can include a shaft 626. The delivery catheter 106 can include a heart valve accommodation region 652. The heart valve accommodation region 652 can be configured to receive and hold a heart valve. In some embodiments, the delivery catheter 106 can also include a balloon. In some embodiments, a balloon is omitted and a frame of the heart valve can be self-expanding. In some embodiments, other mechanical devices can be used to expand the heart valve into position. In some embodiments, the delivery catheter 106 can be configured as a transfemoral delivery catheter.

The delivery catheter 106 can include a release handle 602. The release handle 602 provides an ergonomic fit to the physician’s hand to facilitate the precise two step deployment of the valve. The release handle 602 comprises a first rotating knob 604 acting on an outer member for the controlled release of the proximal section of the valve from the delivery system, a second rotating knob 606 acting on the inner member and capsule for the controlled release of the distal section of the valve from the delivery system, a safety button 610 to avoid premature release of the distal section of the valve, and a female Luer lock 608 / flushing line for flushing the annular space between the inner, middle and outer members.

As part of the implant procedure, radiopaque markers can be used for correct mid-stent positioning. Forward motion of the system can be used for correct leaflet engagement. Deployment can then be executed as a two-step process. In the first step, the first rotating knob 604 can be turned to release the upper crown and stabilization arches. Once the position is verified, then second rotating knob 606 can be turned to release the lower crown for full deployment. In order to retrieve the delivery system, the first step is to ensure that the stent holder releases cleanly from valve. If stuck, then the delivery system is carefully advanced into left ventricle for disengagement. Once the system is in the descending aorta, then the system is closed by reversing the knobs and the system can be withdrawn.

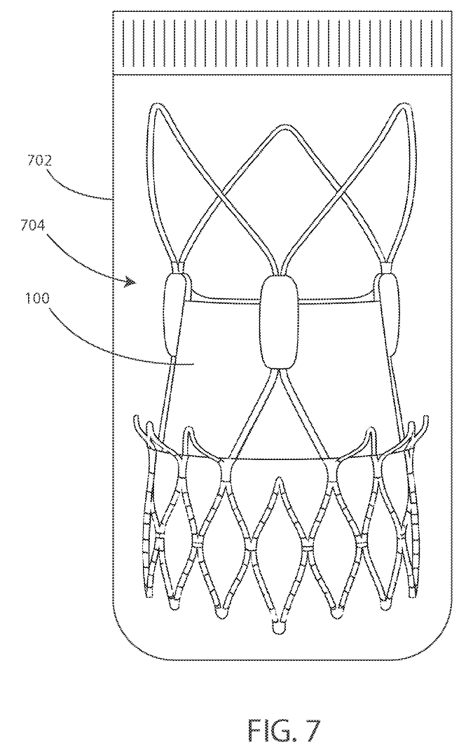

The heart valve replacement system can include a sterile package. It will be appreciated that the package can take on various forms including flexible or non-flexible packages that can include pouches, trays, bags, boxes, cartons, containers, and the like. In some embodiments, the package can be hermetically sealed and can contain an inert gas or air. In some embodiments, the package can be breathable packaging that prevents ingress of bacteria. The heart valve and the delivery catheter can both be disposed within the same package or within separate packages. In some embodiments, other components useful with a valve replacement procedure such as loading tools and a sheath (such as an access sheath or introducer sheath) can also be disposed within the same package or within other packages. Referring now to FIG. 7, a schematic view is shown of a heart valve 100 within a package 702. The package 702 can be hermetically sealed and can contain an inert gas or air. The package 702 can be rigid or flexible. The package 702 can define an interior volume 704. In some embodiments, the interior volume 704 can be sufficiently sized to fit multiple components of the system such as the valve 100, loading tools, and portions of the delivery system such as the delivery catheter. In other embodiments, the interior volume 704 may only be big enough to contain the valve 100. It will be appreciated that the package 702 can be formed of various polymers or other materials.

### Methods

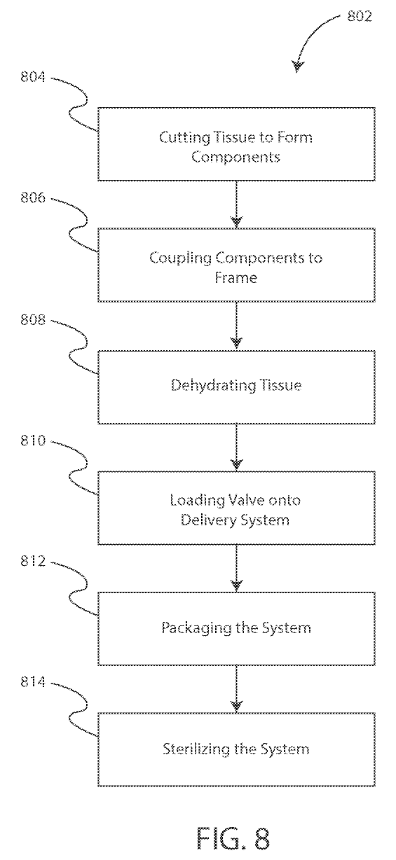

Referring now to FIG. 8, a flowchart depicting a method is shown in accordance with various embodiments herein. FIG. 8 shows a method of making 802 a heart valve replacement device. The method can include an operation of cutting 804 animal tissue into leaflets, skirts, or other components. Other operations can include washing the tissue to remove materials such as blood, fats, and cellular debris. In various embodiments, a decellularization process can be performed. In various embodiments, a fixation operation can be performed, such as by using a glutaraldehyde solution or other cross-linking agents to fix or cross-link the tissue.

The method can include an operation of coupling 806 the leaflets and/or the skirt to a frame to form a heart valve. In various embodiments, such components can be attached to the frame using sutures. The valve frame, in some embodiments, can include apertures therein to aid in coupling the components and/or to aid in suturing.

In various embodiments herein, multiple rinses are performed to remove the glutaraldehyde. By way of example one, two, three or more rinses can be performed using solutions such as a pH buffered solution or a saline solution and/or a saline-glycerol solution. After rinses are complete, the amount of remaining glutaraldehyde can be less than about 2, 1, 0.5, 0.1, 0.05, 0.0025, or 0.0001 weight percent (wt. %). In some embodiments, the amount of remaining glutaraldehyde is only trace amounts or virtually zero.

The method can include an operation of dehydrating 808 moisture from the leaflets, skirt, or other animal tissue. In some embodiments, dehydrating can take place after the operation of coupling 806 the leaflets and/or the skirt to a frame to form a heart valve. While not intending to be bound by theory, it is believed that coupling procedures (such as, but not limited to, suturing) can be executed more effectively if moisture has not yet been removed due to one or more of dimensional changes, material properties, and the like. Dehydrating 808 herein can include various specific operations. In some embodiments, the tissue and/or the tissue attached to the frame can be soaked in a saline-glycerol solution (such as 45% v/v glycerol in saline) for a period of time such as 3, 6, 9, 12, 15, 18, 24, or 36 hours or more, or an amount of time falling within a range between any of the foregoing.

In some embodiments, the tissue and/or the tissue attached to the frame can be dried in an oven. In various embodiments, the oven temperature can be greater than room temperature but not more than 50° C. For example, the oven can be at 37 degrees Celsius for a period of time such as for at least 4, 6, 8, 12, 16, 20, 24, 36, 48, 60, 72, or 84 hours or more, or an amount of time falling with in a range between any of the foregoing.

At the conclusion of the dehydration operations, the valve leaflets remain soft and flexible. The amount of water remaining in the tissues can be less than about 50, 40, 35, 30, 25, 20, 15, 10, or 5 wt. %, or an amount falling within a range between any of the foregoing. In some embodiments, the leaflets and/or skirt can have a final glycerol content of about 30 to about 65 wt. % after removing moisture. In some embodiments, the leaflets and/or skirt can have a final glycerol content of about 15 to about 30 wt. % after removing moisture. In some embodiments, the leaflets and/or skirt can have a final glycerol content of about 40 to about 60 wt. % after removing moisture. In some embodiments, the leaflets and/or skirt can have a final glycerol content of about 40 to about 50 wt. % after removing moisture.

The method can also include an operation of loading 810 the valve on the delivery system. In some embodiments, this can include crimping the valve onto a delivery catheter. In some embodiments, loading 810 the valve onto the delivery system can take place at the time of manufacturing the valve. In other embodiments, loading the valve onto the delivery system may take place near the time of clinical use. The method can also include an operation of packaging 812 the system, such as the heart valve and the delivery catheter.

The method can include an operation of sterilizing 814 the valve. Details of exemplary sterilization techniques are provided in further detail below. The heart valve can be sterilized separately and packaged or can be preloaded on the delivery system and sterilized and then packaged.

It has been found that the sterilization process can reduce the thickness of the valve tissue. In some embodiments, the thickness of the valve tissue after sterilization is less than about 0.010, 0.009, 0.0085, 0.008, 0.0075, 0.007, 0.0065, or 0.006 inches (0.254 mm, 0.229 mm, 0.216 mm, 0.203 mm, 0.191 mm, 0.178 mm, 0.165 mm, or 0.152 mm) or a thickness falling within a range between any of the foregoing.

### Dehydrating Composition

As discussed above, the leaflets and/or the skirt can be contacted or submerged in a dehydrating composition that includes glycerol such as glycerol and saline. In some embodiments, contacting the leaflets and/or the skirt with a composition can include spraying or pouring the composition onto the leaflets. In some embodiments, the leaflets and/or the skirt can be contacted or submerged in the composition multiple times. In some embodiments, the leaflets and/or the skirt are in contact with the composition until the tissue and the composition stabilize, such as obtaining an equilibrium inside the tissue. In some embodiments, the leaflets and/or the skirt are in contact with the composition for at least about 1, 2, 4, 6, 8, 12, 24, 36, 48 hours or more, or an amount of time falling within a range between any of the foregoing.

In various embodiments, the amount of glycerol in the composition can be about 30, 40, 50, 60, 70, 80, 90, 95 or 98 percent by weight, or an amount falling within a range between any of the foregoing. In various embodiments, the amount of water in the composition can be less than about 60, 50, 40, 30, 20, or 10 percent by weight, or an amount falling within a range between any of the foregoing.

In various embodiments, the amount of salt (such as a sodium salt) in the composition can be about 0, 0.1, 0.2, 0.5, 1, 1.5, 2, 2.5, 3, 4, 5, 6, 7, 8, 9, 10, 11, 13, 15, 17, 20, 25, or 30 percent by weight, or an amount falling within a range between any of the foregoing. In some embodiments the amount of salt in the composition can be sufficient such that the composition has a physiologic osmolarity and/or can be isotonic with blood. In various embodiments, the salt content can be provided by an amount of saline or buffered solutions such as phosphate buffered saline.

### Animal Tissue

In various embodiments, the leaflets, the inner skirt, and/or the outer skirt can include an animal tissue. In various embodiments, the animal tissue can include at least one of porcine tissue and bovine tissue. However, tissues from other species could also be used such as equine tissue.

Tissue harvested from the pericardium, from veins, from existing valves, and the like can be used to make valve components herein. However, in some embodiments, the animal tissue is specifically pericardial tissue.

After processing in accordance with operations herein and as attached onto the frame of a valve, the animal tissue can include various amounts of glycerol. In some embodiments, the amount of glycerol can be greater than or equal to 15 wt. %, 20 wt. %, 25 wt. %, 30 wt. %, 35 wt. %, 40 wt. %, 45 wt. %, 50 wt. %, 55 wt. %, or 60 wt. %, or an amount falling within a range between any of the foregoing. In various embodiments, the animal tissue can include less than about 50 wt. %, 45 wt. %, 40 wt. %, 35 wt. %, 30 wt. %, 25 wt. %, 20 wt. %, 15 wt. %, or 10 wt. % water, or an amount falling within a range between any of the foregoing.

In some approaches to reducing calcification, a capping agent (such as an aqueous glycine solution or other amino acid solutions, various amine-containing buffers, reducing agents, or the like) can be utilized to react with and thus neutralize these free aldehyde sites. However, in various embodiments, tissues herein are not treated chemically to cap free aldehydes. As such, in various embodiments, free aldehydes of the animal tissue are substantially uncapped.

### Sterilization

One technique for sterilizing valves herein involves the use of ethylene oxide (EO) gas. This approach is suitable due to EO's ability to penetrate complex geometries and porous materials like those found in heart valve constructs, effectively eliminating microbial contaminants. The EO sterilization process can include preconditioning the heart valve systems by adjusting temperature and humidity levels, followed by EO exposure under controlled conditions. After achieving the necessary sterilization efficacy, an aeration phase can be conducted to eliminate residual EO gas from the construct.

The temperature at which the EO sterilization process is conducted can affect the structural integrity of the heart valve components. If the temperature exceeds the optimal range, it could compromise the mechanical properties of the bioprosthetic material. In certain embodiments, the EO sterilization described herein is performed with a maximum temperature of 50 degrees Celsius.

In addition to using ethylene oxide (EO) for sterilization, ionizing radiation techniques such as X-ray irradiation, gamma irradiation, or electron beam irradiation can also be employed within the scope of herein. Irradiation sterilization can involve exposing the prosthetic heart valve to ionizing radiation to achieve sterilization. The irradiation sterilization process is controlled to ensure thorough sterilization without compromising the integrity of the valve materials. This involves controlling parameters such as exposure time and the dose. In various embodiments herein, such as in the case of X-ray irradiation, the dose can be selected to maintain material properties while ensuring effective sterilization. In some embodiments, the dose can be 15 kiloGray (kGy), 20 kGy, 25 kGy, 30 kGy, 35 kGy, 40 kGy, 45 kGy, 50 kGy, 55 kGy, 60 kGy, or greater or less, or in some embodiments a dose falling within a range between any of the foregoing.

In various embodiments, irradiation associated with ionizing radiation techniques for sterilization herein can take place in an inert gas atmosphere (such as nitrogen, argon, or the like) to reduced oxidative effects from radiation.

Significantly, as shown in example 1 below, it has been discovered herein that prosthetic heart valves fabricated in accordance with embodiments herein and, particularly related to their sterilization process, exhibit remarkable anti-calcification properties. For example, by employing a specific sterilization protocol involving ethylene oxide (EO) exposure, a notable resistance to calcific degradation has been observed, distinguishing these prosthetic valves from conventional counterparts.

Similarly, the use of irradiation has been found to provide benefits regarding calcification reduction. The irradiation process, involving precisely moderated doses of x rays, appears to reduce the formation of calcific deposits on the leaflet tissues and other tissues of the valve.

While not intending to be bound by theory, it is believed that dehydration and then sterilization as described herein makes the tissue thinner and more dense and that such changes function to impede the nucleation and growth of calcium deposits through mechanisms such as by reducing diffusion of calcium into the tissue and/or blocking binding of calcium compounds through steric hinderance, thereby impeding calcification processes and preserving the valve's mechanical compliance and operational longevity.

In various embodiments herein, the tissue exhibits deposition of less than 35,000, 30,000, 25,000, 20,000, 18,000, 16,000, 15,000, 14,000, 13,000, or 12,000 micrograms of calcium per gram of tissue after twelve weeks in a calcifying solution, or an amount of calcium deposition falling within a range between any of the foregoing.

Aspects may be better understood with reference to the following examples. These examples are intended to be representative of specific embodiments, but are not intended as limiting the overall scope of embodiments herein.

### EXAMPLES

### Example 1: In Vitro Calcification Study

Four tissue materials were prepared (porcine pericardium). In specific, the sample tissues were as follows: tissue “A” was first processed by fixing the tissue with a glutaraldehyde solution, rinsing three times with a saline solution, dehydrating using a 45% by volume glycerol and saline solution, drying in an oven at 37 degrees Celsius for over 8 hours, and then sterilizing with a dose of approximately 50 kGy of X-ray irradiation; tissue “B” was processed the same as for A, but sterilized with EO; tissue “C” was a control tissue representing a standard valve porcine pericardium wet (non-dehydrated) tissue treated with a standard anti-calcification treatment; and tissue “D” was a control in the form of a porcine pericardium tissue with no anti-calcification treatment.

The four tissue treatments were then evaluated for calcification formation by immersing them in a concentrated calcium phosphate solution that was refreshed weekly for twelve weeks. The tissues were then removed and evaluated for calcification using inductively coupled plasma optical emission spectroscopy (ICP-OES). Briefly, samples were weighed and acid digested. Calcium concentrations were calculated using a standard curve.

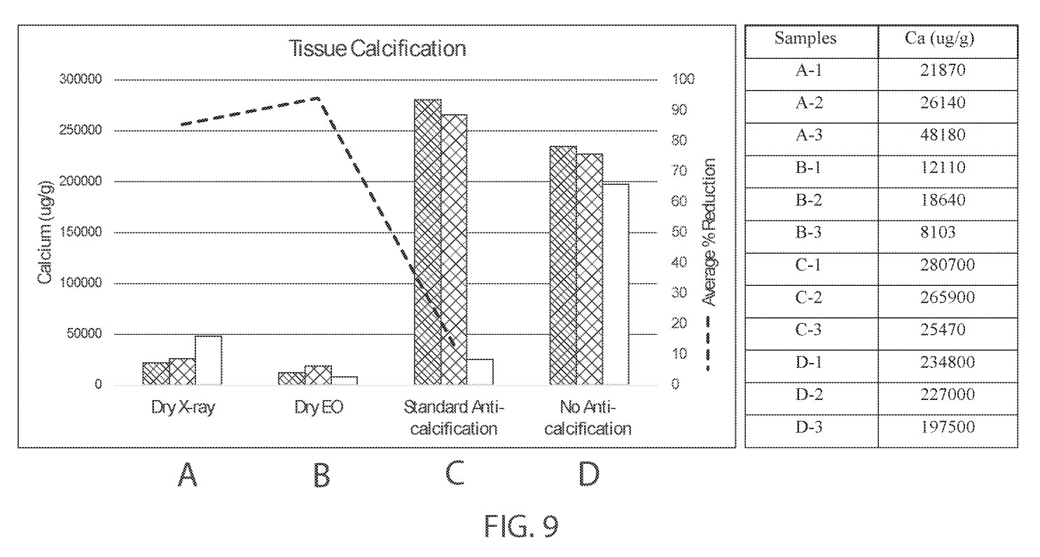

The results are shown in FIG. 9. The results demonstrate that after 12 weeks in a highly calcifying solution, tissues “A” and “B” had minimal calcification and, in particular, significantly less than “C” and far less than “D” (untreated control tissue). For example, tissue “A” (x-ray irradiation) exhibited calcification reduction of 85.4% compared with “D” (untreated control tissue). Tissue “A” (x-ray irradiation) performed even better and exhibited calcification reduction of greater than 94.1% compared with “D” (untreated control tissue).

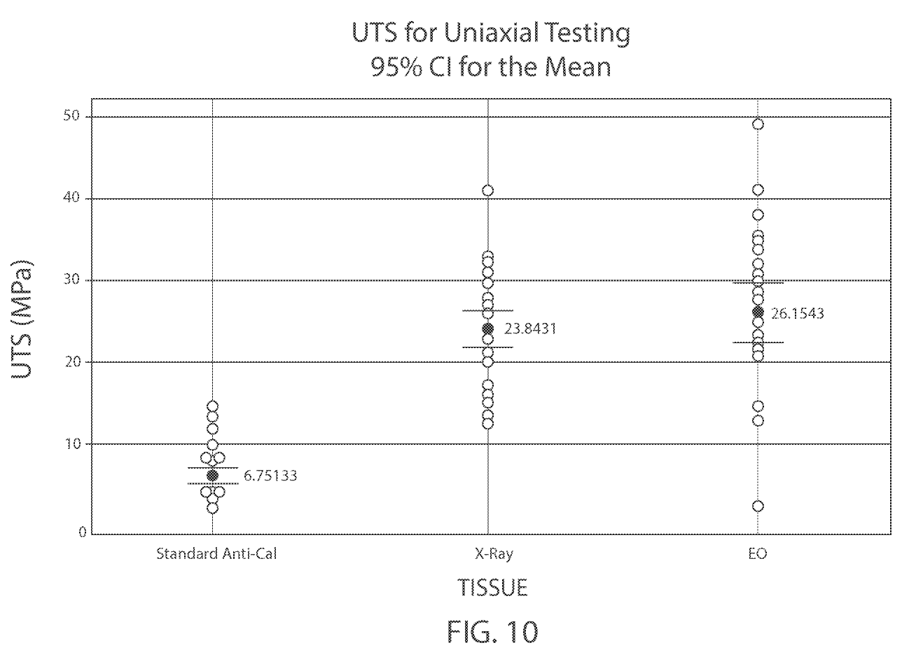

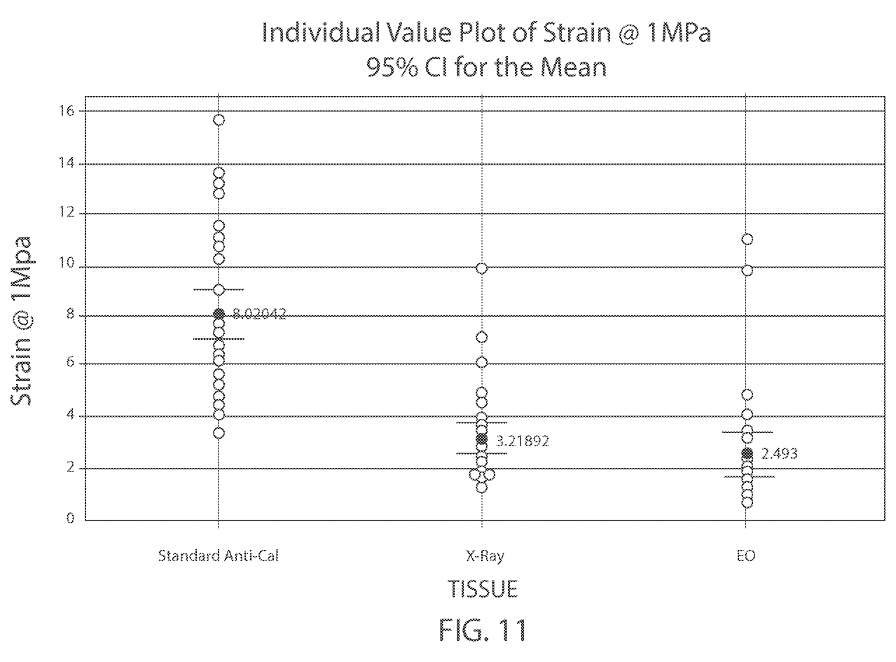

Tissue thicknesses were measured before drying and sterilization and again after rehydrating and it was found that even though tissues “A”, “B”, “C”, and “D” started off at the same thickness, tissues “A” and “B” were thinner than tissues “C” and “D”, both having an average thickness of less than 0.008 inches (or 0.203 millimeters). The tissues were also evaluated for tensile strength using uniaxial tensile testing. Samples were rehydrated and prepared by cutting uniaxial dogbones from the given tissue following dimensional recommendations from ASTM D412 (testing of vulcanized rubber and thermoplastic elastomers). Samples were preconditioned from 0.1 – 1.0 MPa for 5 cycles, then pulled in strain-to-failure, where ultimate tensile stress (UTS) (illustrated in FIG. 10) and strain at 1MPa (illustrated in FIG. 11) were calculated using the resulting loads and the tissue thickness for each sample. It was found that dry sterilized tissues exhibited increased tensile strength and stiffness in comparison with standard, wet anti-calcification treated tissue. X-Ray tissue exhibited a UTS of 23.84 MPa, EO tissue exhibited a UTS of 26.15 MPa, and standard anti-calcification tissue exhibited a UTS of 6.75MPa.

Furthermore, X-Ray tissue exhibited a strain at 1MPa of 3.2%, EO tissue exhibited a strain at 1MPa of 2.49%, and standard anti-calcification tissue exhibited a strain at 1MPa of 8.02%. These results confirm that dry sterilized tissue exhibited an increase in stiffness, as less strain was required to reach 1MPa of stress compared to standard anti-calcification tissue.

These results highlight the added benefit of increased durability for dry sterilized tissue herein (such as tissues “A” and “B”), as samples showed an increase in UTS and stiffness.

In summary, this example shows that tissues treated in accordance with embodiments herein exhibit surprisingly high resistance to calcification. In specific, this example shows that the combination of dehydration and sterilization as described herein provides surprising anti-calcification effects. This example further shows that tissues processed as described herein exhibited high levels of tensile strength while maintaining sufficient flexibility to perform well as valve leaflets.

It should be noted that, as used in this specification and the appended claims, the singular forms "a," "an," and "the" include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase "configured" can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A prosthetic valve system with anti-calcification properties comprising:
a prosthetic valve, the prosthetic valve comprising
a frame; and
a plurality of valve leaflets, wherein the plurality of valve leaflets are coupled to the frame, the valve leaflets comprising
an animal tissue, the animal tissue comprising
from 15% to 50% by weight water;
from 20% to 70% by weight glycerol; and
less than 0.01 percent by weight glutaraldehyde;
a package, wherein the prosthetic valve is disposed within an interior volume of the package without glutaraldehyde;
wherein the animal tissue has an average thickness less than or equal to 0.012 inches thick in a sterilized state as disposed within the package; and
wherein the animal tissue exhibits anti-calcification properties that are greater than otherwise similar tissue that has not been sterilized within the package.

2. The prosthetic valve system with anti-calcification properties of claim 1, further comprising a delivery catheter.

3. The prosthetic valve system with anti-calcification properties of claim 1, wherein the animal tissue is porcine pericardium.

4. The prosthetic valve system with anti-calcification properties of claim 1, the prosthetic valve further comprising an aortic valve.

5. The prosthetic valve system with anti-calcification properties of claim 1, the prosthetic valve further comprising a transcatheter aortic valve.

6. The prosthetic valve system with anti-calcification properties of claim 1, the prosthetic valve further comprising an inner skirt;
wherein the inner skirt is attached to the frame; and
wherein the inner skirt is formed of an animal tissue, the animal tissue comprising
from 15% to 50% by weight water;
from 20% to 70% by weight glycerol; and
less than 0.01 percent by weight glutaraldehyde.

7. The prosthetic valve system with anti-calcification properties of claim 1, the prosthetic valve further comprising an outer skirt;
wherein the outer skirt is attached to the frame; and
wherein the outer skirt is formed of an animal tissue, the animal tissue comprising
from 15% to 50% by weight water;
from 20% to 70% by weight glycerol; and
less than 0.01 percent by weight glutaraldehyde.

8. The prosthetic valve system with anti-calcification properties of claim 1, wherein free aldehydes of the animal tissue are uncapped.

9. The prosthetic valve system with anti-calcification properties of claim 1, wherein the animal tissue is less than or equal to 0.008 inches thick in a sterilized state as disposed within the package.

10. The prosthetic valve system with anti-calcification properties of claim 1, wherein the animal tissue exhibits deposition of less than 35,000 micrograms of calcium per gram of tissue after 12 weeks in a calcifying solution.

11. A method of making a heart valve with anti-calcification properties comprising:
fixing tissue with a glutaraldehyde solution;
attaching the tissue to a frame to form valve leaflets;
dehydrating the tissue;
packaging the heart valve; and
sterilizing the heart valve.

12. The method of making a heart valve with anti-calcification properties of claim 11, further comprising rinsing the tissue to remove glutaraldehyde prior to dehydrating the tissue.

13. The method of making a heart valve with anti-calcification properties of claim 11, wherein dehydrating the tissue comprises:
soaking the tissue in a glycerol and saline solution; and
drying the tissue in an oven.

14. The method of making a heart valve with anti-calcification properties of claim 11, wherein sterilizing the heart valve comprises at least one selected from the group consisting of ethylene oxide (EO) sterilization and irradiation, wherein irradiation takes place in an inert gas atmosphere to reduce oxidative effects from radiation.

15. The method of making a heart valve with anti-calcification properties of claim 14, wherein sterilization comprises ethylene oxide (EO) sterilization with a maximum temperature of 50 degrees Celsius or X-ray irradiation with dosage of 15 to 60 kGy.

16. The method of making a heart valve with anti-calcification properties of claim 11, wherein sterilizing the heart valve reduces a thickness of the leaflet tissue by at least 5 percent.

17. The method of making a heart valve with anti-calcification properties of claim 11, wherein the tissue exhibits anti-calcification properties that are greater than otherwise similar tissue that has not been sterilized after dehydration and packaging.

18. The method of making a heart valve with anti-calcification properties of claim 11, wherein the tissue is porcine pericardium.

19. The method of making a heart valve with anti-calcification properties of claim 11, wherein the heart valve is an aortic valve.

20. The method of making a heart valve with anti-calcification properties of claim 11, wherein the tissue exhibits deposition of less than 35,000 micrograms of calcium per gram of tissue after 12 weeks in a calcifying calcium phosphate solution.
